(19) United States
(12) Patent Application Publication
(10) Pub. No.: US 2004/0048237 A1
(43) Pub. Date: Mar. 11, 2004

(54) Mammalian prion proteins and transgenic mice expressing them

(76) Inventors: Susan L. Lindquist, Chestnut Hill, MA (US); Jiyan Ma, Chicago, IL (US)

Correspondence Address: ROPES & GRAY LLP, ONE INTERNATIONAL PLACE, BOSTON, MA, 02110-2624, US

(21) Appl. No.: 10/438,628

(22) Filed: May 15, 2003

Related U.S. Application Data
(60) Provisional application No. 60/380,953, filed on May 15, 2002.
(60) Provisional application No. 60/380,950, filed on May 15, 2002.
(60) Provisional application No. 60/419,574, filed on Oct. 17, 2002.
(60) Provisional application No. 60/419,569, filed on Oct. 17, 2002.

(51) Int. Cl. C12Q 1/00; G01N 33/53; G01N 33/567

(52) U.S. Cl. 435/4; 435/7.2

(57) ABSTRACT

The invention relates to methods of identifying, detecting or designing drugs that inhibit the formation of or accumulation of PrP, PrP^{Sc} or both in cells; to drugs that inhibit the formation of or accumulation of PrP, PrP^{Sc} or both in cells; to methods of preventing or reducing the adverse effects of PrP, PrP^{Sc} or both in humans; and to transgenic nonhuman mammals, such as transgenic mice, that ectopically express PrP.

### RELATED APPLICATIONS

This application claims the benefit of the filing dates of U.S. Provisional Application No. 60/380,953, entitled “Mammalian Prion Proteins”, by Susan Lindquist and Jiyan Ma (filed May 15, 2002); U.S. Provisional Application No. 60/380,950, entitled “Transgenic Mice Expressing Prion Protein”, by Susan Lindquist and Jiyan Ma (filed May 15, 2002); U.S. Provisional Application No. 60/419,574, entitled “Mammalian Prion Proteins”, by Susan Lindquist and Jiyan Ma (filed Oct. 17, 2002); and U.S. Provisional Application No. 60/419,569, entitled “Transgenic Mice Expressing Prion Protein”, by Susan Lindquist and Jiyan Ma (filed Oct. 17, 2002). The entire teachings of the referenced Provisional Applications are incorporated herein by reference.

### FUNDING

Work described herein was supported, in whole or in part, by National Institutes of Health Grant No. GM 25874. The United States Government has certain rights in the invention.

### BACKGROUND OF THE INVENTION

Prion diseases are rare fatal neurodegenerative disorders that have the unusual property of appearing in sporadic, dominantly heritable, and transmissible forms(9). Interest in prion diseases has been intensified by the recent outbreak of mad-cow disease, the increasing incidence of the new variant Creutzfeldt-Jakob Disease in young people in Great Britain, and the recent spread of Deer and Elk Wasting Disease in the United States(9,10). Such incidents pose a threat to both the global economy and human health.

Changes in the trafficking and conformation of PrP, the mammalian prion protein, are associated with this group of fatal neurodegenerative diseases, the prion diseases (1). Some of these diseases involve an infectious agent and a large body of evidence supports the remarkable hypothesis that this agent is a protein, specifically, an altered conformation of PrP known as PrP^{Sc}. PrP^{Sc} is thought to propagate by converting other PrP molecules to the same conformation (1). Alternatively, PrP^{Sc} may propagate by association with an as yet unidentified infectious agent (2). In either case, conversion of PrP to PrP^{Sc} is a central event in the development of the transmissible forms of these diseases, yet the mechanism by which conversion is initiated remains a complete mystery.

These diseases have unusually complex etiologies and decades of research have failed to elucidate the pathogenic mechanism (11, 15). A large body of elegant work, however, has established a pivotal role for the prion protein, PrP (16). Increasing evidence suggests that PrP^{Sc} is not itself neurotoxic. PrP^{Sc} is not observed in several inherited and experimentally induced forms of prion disease (17, 23). Moreover, PrP knockout mice are immune to the toxic effects of PrP^{Sc}, even when they receive high titers of PrP^{Sc} intra-cerebrally (16). Various changes in PrP metabolism have been associated with pathogenesis, but which, if any, cause cell death remains a mystery.

### SUMMARY OF THE INVENTION

The present invention relates to mammalian prion proteins and to transgenic mice expressing prion proteins. In one embodiment, the invention relates to the presence of the prion protein, PrP, in the cytoplasm of isolated cells, for example, isolated neuronal cells.

A rare conformation of PrP, PrP^{Sc}, is found only in mammals with transmissible prion diseases and is either the infectious agent itself or a major component of it. Until this time, the mechanism for initiating PrP^{Sc} formation remained unknown. As described herein, when retrograde-transported PrP accumulates in the cytoplasm, it can spontaneously convert to a PrP^{Sc}-like conformation. As also described herein, conversion is nonlinear with concentration: cytoplasm PrP forms amorphous aggregates unless it accumulates at a sufficient rate to convert to the PrP^{Sc}-like state. Once conversion occurs, it is maintained, demonstrating that PrP has an inherent capacity to promote its own conformational conversion in mammalian cells.

Applicants have shown that the presence of mammalian prion protein, PrP, in the cytoplasm of a cell is sufficient to kill (is toxic to) the cell. They have shown that cytoplasmic accumulation of small amounts of PrP^{Sc} is selectively neurotoxic. Further, they have shown that when PrP accumulates in the cytoplasm, it can spontaneously convert to an altered conformation of Prp, PrP^{Sc}, referred to as a protein associated with a group of fatal neurodegenerative diseases, the prion diseases. In addition, Applicants have shown that once conversion begins, it continues, thus showing for the first time that it has a self-sustaining character and providing a model to explain the spontaneous formation or origin of PrP^{Sc}.

When proteosome activity is compromised, PrP accumulates in the cytoplasm and the concentration of the PrP required for formation of the altered prion protein, PrP^{Sc}, is more likely to be reached and, thus it is more likely that, when cells containing even a very small amount of PrP die, PrP^{Sc} will be released to propagate through its normal infectious cycle.

This invention relates to methods of identifying or designing drugs (agents), which can be compounds or molecules, that inhibit formation (production) of or accumulation of PrP, PrP^{Sc} or both in cells, particularly in the cytoplasm of cells, and, thus, reduce cytotoxicity of these prion proteins; reduce release of PrP^{Sc} from cells; and/or reduce PrP^{Sc} infectivity. The methods of the present invention, thus, are methods of identifying, detecting or designing drugs that reduce (totally or partially) the adverse effects of neurodegenerative diseases in which PrP, PrP^{Sc} or both play a role, particularly neurodegenerative prion protein diseases, including fatal neurodegenerative prion protein diseases. In one embodiment, the invention is a method of identifying a drug that inhibits formation of (e.g., the presence of) PrP in mammalian cells, comprising culturing test cells in the presence of a candidate drug, wherein test cells (e.g., mammalian, such as mouse or human) are cells that ectopically express PrP in the cytoplasm and comparing viability of the test cells with viability of control cells, wherein if viability of test cells cultured in the presence of the candidate drug is greater than viability of control cells, the candidate drug is a drug that inhibits formation of PrP in mammalian cells. Control cells are the same as test cells, but are cultured in the absence of the candidate drug. Control cells can be cultured simultaneously with the test cells or can be cultured at a different time (e.g. prior to or after test cells are cultured) and results used to produce a reference or standard with which results obtained with test cells can be compared. Culturing cells in the presence of a candidate drug includes contacting cells with the candidate drug such as, for example, contacting cells with a candidate drug under conditions suitable for ectopic expression of PrP in the cells. The viability of cells refers to cell survival. Comparing the viability of cells includes comparing the percent of cell survival between two cell populations (e.g., test cells and control cells). A cell population may consist of one or more than one cell. Viability includes determining the number of cells that survive (e.g., the percent of test cells that can continue to be cultured under test conditions in comparison to the percent of control cells that can continue to be cultured under test conditions). Conversely, cell viability can be determined by comparing the extent of cell death between two cell populations (e.g., the number of test cells that die in comparison to the number of control cells that die).

In an additional embodiment, the invention relates to a method of identifying a drug that inhibits (reduces or prevents) the accumulation of PrP^{Sc} in mammalian cells, such as mouse or human neuronal cells, comprising culturing test cells in the presence of a candidate drug, wherein test cells (e.g., mammalian, such as mouse or human) are cells that ectopically express PrP^{Sc} in the cytoplasm and comparing viability of the test cells with viability of control cells, wherein if viability of test cells cultured in the presence of the candidate drug is greater than viability of control cells, the candidate drug is a drug that inhibits accumulation of PrP^{Sc} in mammalian cells. In a further embodiment, the invention relates to a method of identifying a drug that inhibits the presence of PrP^{Sc} or PrP in the cytoplasm of mammalian cells. In an additional embodiment, the invention relates to a method of identifying a drug that inhibits the formation of a (one or more) pathological conformation of PrP in mammalian cells. In a further embodiment, the invention relates to a method of identifying a drug that inhibits improper processing (misfolding and/or incorrect localization) of PrP in mammalian cells. In yet another embodiment, the invention relates to a method of identifying a drug that inhibits PrP toxicity in mammalian cells. In these embodiments, nucleic acids (e.g., DNA or RNA) encoding PrP to be expressed in the cytoplasm of cells are under the control of regulatory element(s), such as a promoter (e.g., a tetracycline-inducible promoter or an ecdysone-inducible promoter), which enables expression of PrP in the cytoplasm. Expression can be constitutive or inducible and an appropriate promoter (and, optionally, additional regulatory elements such as, for example, an enhancer) can be used.

This invention further relates to drugs identified, detected or discovered by the present methods, as well as to drugs that inhibit formation (e.g., the appearance or presence) or accumulation of PrP, PrP^{Sc} or both in cells. It further relates to methods of reducing PrP formation or accumulation in cells, particularly in mammalian cells, such as human cells. Thus, the present method also relates to methods of preventing or reducing (partially or totally) the adverse effects of PrP, PrP or both in individuals, particularly humans who have or could develop a prion protein disease, such as a neurodegenerative disease. In one embodiment of the method, at least one drug that reduces (partially or totally) PrP formation and/or accumulation in the cytoplasm of cells, particularly neuronal cells, is administered to an individual in need of treatment for such a disease. In another embodiment, at least one drug that reduces (partially or totally) PrP^{Sc} formation in the cytoplasm of cells is administered to an individual in need of treatment. In a further embodiment, at least one drug that reduces PrP appearance and/or accumulation in the cytoplasm and at least one drug that reduces PrP^{Sc} formation in the cytoplasm are administered, simultaneously or sequentially, to the individual. In another embodiment, at least one drug that reduces both PrP formation (e.g., appearance or presence) and/or accumulation in the cytoplasm as well as PrP^{Sc} formation in the cytoplasm is administered to an individual in need of treatment. In a further embodiment, at least one drug that reduces the formation of a (one or more) pathological conformation of PrP in the cytoplasm of cells is administered to an individual in need of treatment. In an additional embodiment, at least one drug that reduces PrP toxicity in mammalian cells (e.g., mouse or human cells) is administered to an individual in need of treatment. In all embodiments, the drugs can be administered with other drugs or forms of therapy, such as a component of a “cocktail” of drugs. A drug that interferes with processing of the proteins (e.g., unfolding during retrograde transport) that would make them available for conversion to or formation of PrP^{Sc} can be given, alone or in combination with other drugs.

Applicants herein have shown that the accumulation of even small amounts of cytoplasmic PrP is strongly and selectively neurotoxic in cultured cells and in transgenic mice. Mice develop normally but acquire severe ataxia, with cerebellar degeneration and gliosis. This identification of a toxic species of PrP suggests a common mechanism for seemingly disparate PrP-related neuropathies and has important implications for public health.

Another embodiment of the present invention is a transgenic mouse or other nonhuman mammal that ectopically expresses PrP in the cytoplasm of cells. The PrP expressed is encoded by nucleic acids (DNA or RNA) introduced into at least one cell (a cell or cells) from which the transgenic mouse or an ancestor thereof was produced. In one embodiment, the transgenic mouse is heterozygous for the nucleic acid (e.g., a gene or cDNA) that encodes PrP to be expressed in the cytoplasm. In a second embodiment, the transgenic mouse is homozygous for the nucleic acid (e.g., a gene or cDNA) that encodes PrP. Alternatively, PrP can be expressed from an endogenous gene whose expression is enhanced (turned on or increased from a lower expression level) by known methods. In both embodiments, nucleic acids encoding PrP to be expressed in the cytoplasm of cells are under the control of regulatory element(s), such as a promoter, which enables expression of PrP in the cytoplasm. Expression can be constitutive or inducible and an appropriate promoter (and, optionally, additional regulatory elements) is used for each. For example, nucleic acids (e.g., DNA) encoding the mature form of the wild-type PrP, such as that described in Example 3 or an equivalent thereof, can be placed under the control of an appropriate promoter (see, e.g., Example 3 and references 54 and 55). This construct has been used to produce transgenic mice. Such transgenic mice and their progeny are a subject of this invention. Heterozygous mice, in which the neurodegenerative disease progresses slowly, and homozygous mice, in which the diseases progress rapidly, are both useful for assessing the effects of drugs and changes in environmental or cellular conditions on progression of the disease. Heterozygous transgenic animals can be maintained and bred to produce offspring. Homozygous transgenic offspring are particularly useful for assessing the effects of candidate therapies (e.g., drugs) on the disease, since the condition progresses rapidly. A particular embodiment of the present invention is a transgenic mouse expressing mature PrP, either constitutively or in an inducible manner, in the cytoplasm of its cells.

Also the subject of this invention is a method of identifying, detecting or designing a drug that reduces (totally or partially) the effects of PrP expression and/or appearance or accumulation in the cytoplasm of cells (e.g., neuronal, CNS cells). In one embodiment of the invention, the invention relates to a method of identifying a drug that inhibits (reduces or prevents) the effects of PrP present in the cytoplasm of cells, comprising administering a candidate drug to a test animal, such as a transgenic nonhuman mammal that ectopically expresses PrP in the cytoplasm of its cells, and assessing the effects of PrP on the test animal and assessing the effects of PrP on a control animal, wherein if the effects of PrP in the test animal are less than the effects of PrP in a control animal, then the candidate drug is a drug that inhibits the effects of PrP present in the cytoplasm of cells. A control animal is the same as a test animal except that it is not exposed to the candidate drug.

### BRIEF DESCRIPTION OF THE DRAWINGS

FIGS. 1A-1E show the state of PrP in cells exposed to proteasome inhibitors. PrP was detected by immunoblot analysis using 3F4 antibody or antibodies specific for the C-terminal region (R20) of mature PrP, as indicated.

FIG. 1A: Supernatant (Sup) and pellet fractions of detergent lysates from control cells (C) or cells treated with proteasome inhibitors for 16 hours (epoxomicin, MG132, (MG), or Lactacystin, (Lac)). TT, transient transfection; ST, stable transfection.

FIG. 1B: Proteinase K digestion (20 μg/ml. for 30 min. at 37° C.) of total cell lysates from (A).

FIG. 1C: PK-resistant PrP fragments reacted with 3F4 and R20 antibodies.

FIG. 1D: Time course of PrP accumulating in the pellet fraction of COS cells after proteasome inhibitor treatments. Two independent experiments are shown, each exposed to provide the best comparison of early samples within the experiment.

FIG. 1E: COS cells were transfected with wild-type PrP (WT) or the D177N mutant (M) and treated with epoxomicin.

FIGS. 2A-2C present results that demonstrate that transient proteasome inhibition is sufficient to initiate sustained PrP conversion. COS cells expressing wild-type PrP incubated with or without 50 μM MG132 for 4 hr (2A and 2B). After washing, cells were cultured in normal media for indicated times.

FIG. 2A: Top, immunoblot analysis of co-transfected β-galactosidase confirmed equal levels of transfection. Cell lysates were fractionated by centrifugation (middle: supernatant and pellet fractions analyzed on same gel and exposed for same time) or digested with PK (bottom, exposed 5 times longer).

FIG. 2B: Identical aliquots of the same culture were incubated for the indicated times after the removal of MG132, lysed with detergents and subjected to centrifugation or PK digestion. PrP, P53, β-actin and calreticulin in the same samples were detected with specific antibodies. FIG. 2C: COS cells co-transfected with PrP and CFTR. After transient treatment with DMSO, 1 μM or 5 μM MG132, cells were collected immediately or after 12 hours of culture. PrP and CFTR in the pellet fractions were detected by 3F4 or anti-CFTR antibody.

FIG. 3 is a schematic of a model for spontaneous initiation of PrP^{Sc} in sporadic and inherited forms of prion disease. PrP matures through the ER and appears on the plasma membrane. A portion fails to fold properly and is targeted by the quality control system for retrograde transport to the cytoplasm and proteasomal degradation. If a sufficient number of susceptible species interact they undergo nuclear conversion to the PrP^{Sc} conformation. Other cytoplasmic species of PrP are toxic and kill neurons. The appearance of PrP^{Sc} in the extracellular space leads to propagation.

FIGS. 4A-4F present results that demonstrate the toxicity of cytoplasmic PrP.

FIG. 4A. N2A and WtPN2A cells were treated with MG132 for various times as indicated. Apoptotic cells were identified by TUNEL assay (TdT—mediated dUTP-X nick end labeling, an indicator of apoptosis).

FIG. 4B. WtPN2A cells treated with or without MG132 for 16 hrs were either harvested immediately or cultured to confluence (7 days). PrP in supernatant (s) and pellet (p) fractions was detected.

FIG. 4C. N2A cells stably transfected with presenilin1 (PS1) were treated as in FIG. 4A. Arrows indicated full length PS1, or an NH2-terminal fragment, PS1NT.

FIG. 4D. Different modification states of PrP in WtPN2A and MoPrP cells detected as differences in electrophoretic mobility.

FIG. 4E. MoPrP cells were treated as in FIG. 4A.

FIG. 4F. Immunoblot detection of PrP in N2A cells stably expressing ecdysone-inducible wtPrP or cyPrP. Arrows, PrP; Asterisks, specific PrP cleavage products.

FIGS. 5A-5B depict the nucleic acid sequence of full-length mouse PrP (SEQ ID NO: 1).

FIG. 6 depicts the amino acid sequence of full-length mouse PrP (SEQ ID NO: 2).

FIG. 7 is a schematic of the pCB6+ vector.

### DETAILED DESCRIPTION OF THE INVENTION

This invention relates to methods of identifying or designing drugs (agents), which can be compound or molecules, that inhibit formation (production) of or accumulation of PrP, PrP^{Sc} or both in cells, particularly in the cytoplasm of cells, and, thus, reduce cytotoxicity of these prion proteins; reduce release of PrP^{Sc} from cells; and/or reduce PrP^{Sc} infectivity.

In another embodiment, the invention relates to transgenic mice ectopically expressing PrP.

When PrP is expressed in the cytoplasm of yeast cells, it converts to a conformation with the biochemical properties of PrP^{Sc}, suggesting that some features of this compartment (e.g. chaperones, a reducing environment) might promote conversion of PrP to the rare conformer associated with disease (3). Applicants have described a natural route by which PrP appears in the cytoplasm of mammalian cells. Like many other proteins that mature through the secretory compartment, a substantial portion of PrP is triaged by the endoplasmic reticulum (ER) quality-control system and delivered to the cytoplasm for degradation by the proteasome (4). When proteasome activity is blocked, PrP accumulates in this compartment where it associates with, and causes a massive re-localization of, Hsc70 (4). Applicants reasoned that if the number of molecules delivered to the cytoplasm overwhelms the capacity of various protein quality-control systems of that compartment, some PrP might convert to the PrP^{Sc} conformation.

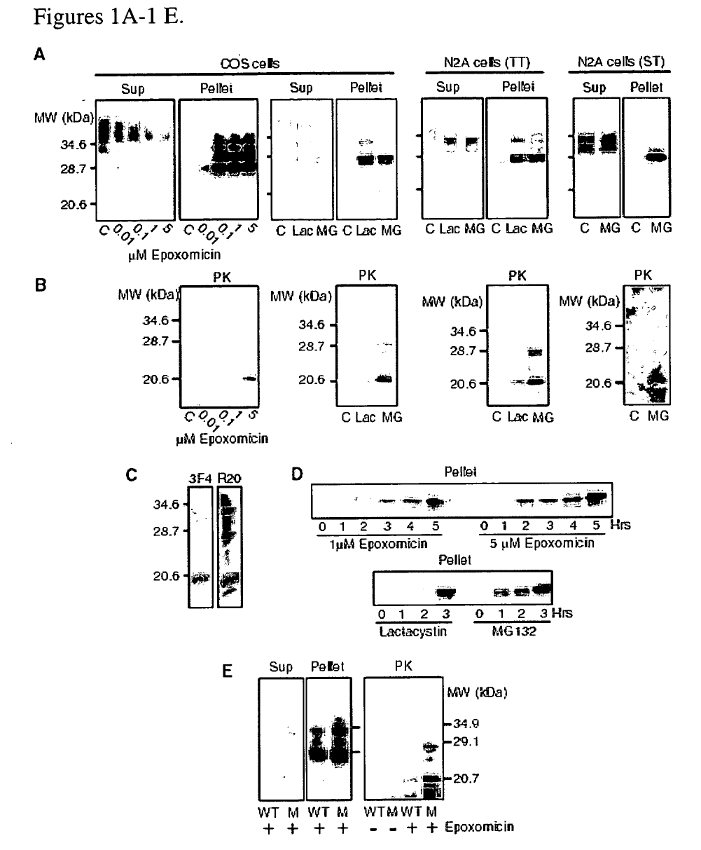

Spontaneous prion diseases are very rare (occurring in about 1/million individuals per year) and even in individuals carrying the most virulent PrP mutations several decades elapse before chance and circumstances produce conversion. As described herein, Applicants increased the likelihood of detecting conversion events by assessing the conformational state of the PrP protein that accumulates in the cytoplasm when proteasome activity is compromised (FIG. 1 and ref. 5). Several cell types were transfected with a wild-type mouse PrP gene, treated with one of three different proteasome inhibitors, lysed with detergents, and subjected to centrifugation. PrP was not detectable before or after proteasome inhibition by Coomassie-blue staining of electrophoretically separated proteins, nor was any other change in the total protein profiles visible (5).

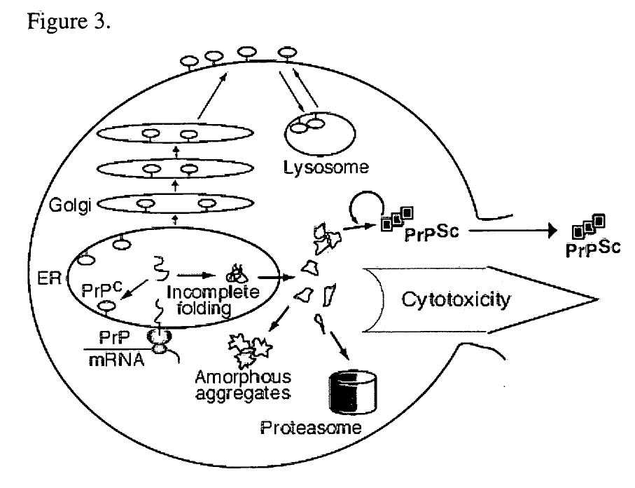

Described herein is an efficient mechanism for converting PrP to a PrP^{Sc}-like state in mammalian cells that involves a natural, continuously occurring process, the retrograde transport of misfolded PrP to the cytoplasm. These findings lead to a model to explain the spontaneous origin of PrP^{Sc} and certain other puzzling features of PrP diseases (FIG. 3). The flux of PrP into the cytoplasm is likely to be influenced by stress, physical trauma, toxins and aging, and is certainly influenced by the presence of disease-associated mutations in PrP (4, 21). When PrP reaches the cytoplasm it has a small but finite chance of converting to the PrP^{Sc} conformation. Even small differences in the rate of its appearance in this compartment can profoundly influence the likelihood of conversion. This, together with direct effects that PrP mutations may have on folding, could readily account for the fact that some mutations reproducibly lead to the production of PrP^{Sc} and others do not.

Once conversion of PrP begins, it has a self-sustaining character, influencing yet more PrP proteins to adopt the same form. This is the first time that the induction of a PrP^{Sc}-like conformation with this critical property has been achieved de novo in living cells. It might be infectious on its own or associate with a latent infectious agent. In either case, its unusual ability to promote an increase in its own concentration seems likely to contribute to the progression and propagation of transmissible forms of PrP diseases. It is the cytoplasmic accumulation of very small amounts of PrP that seems to be selectively neurotoxic, not the appearance of large aggregates or even the PrP Sc conformation. A dissociation between PrP^{Sc} and toxicity has also been observed by others (22). However, if cells do contain PrP^{Sc}, when they die it will be released to propagate through its normal infectious cycle. Many features of the retrograde-transported protein might influence the initial conversion event, but not be required for propagation. Thus, the PrP^{Sc} that accumulates later, during the natural progression of disease, would be expected to include oxidized and glycosylated species.

Under normal circumstances the appearance of PrP^{Sc} is very rare. Even when stress, trauma, aging, or mutations increase retrograde transport and compromise proteasome activity, the extreme toxicity of very small quantities of cytoplasmic PrP would be likely to kill neurons before PrP^{Sc} can form. Indeed, the extreme and highly selective toxicity of cytoplasmic PrP might be an evolved mechanism that generally acts to prevent the formation of potentially infectious material in those cells that are most likely to experience conversion because they express PrP at the highest level, neurons.

Given that proteasome inhibitors are widely used in research and are being employed in the development of cancer and AIDS therapies (24), the present work has important implications for human health. Caution should be taken in research environments and the clinical consequences of proteasome inhibition should be evaluated over the long term. The inhibitors have the potential to generate neurotoxic cytoplasmic forms of PrP. Moreover, since conversion can occur in non-neuronal cells, even peripheral exposures might produce infectious material. The lower concentration of PrP in non-neuronal tissues makes such conversions unlikely, but even a small risk of generating infectious material is most unwelcome.

In another embodiment of the invention, the invention relates to transgenic mice expressing PrP. The perplexities common in PrP research are exemplified by recent work on transmembrane forms (25). PrP is normally a plasma membrane protein, anchored via a GPI linkage, but a small percentage of PrP molecules adopt a transmembrane state (26). Some mutations in the transmembrane region which increase the likelihood that PrP molecules will assume this topology cause neurodegeneration in transgenic mice (25). However, the majority of mutations associated with inherited forms of prion disease are not near the transmembrane region (9), and no change in membrane topology is observed with these mutants (27).

Applicants describe a relationship between PrP misfolding, proteasome inhibition, the accumulation of PrP in the cytoplasm, and selective neurotoxicity that contribute to an explanation of some of these perplexities. They were led to examine this relationship by several observations. First, a substantial fraction of most proteins that traffic through the ER misfold and are routinely retrograde transported to the cytoplasm for degradation by the proteasome (28). Second, both mutant and wild-type PrPs follow this pathway (29, 30). Third, several mutant PrPs associated with familial prion diseases are as stable as wild-type PrP once they have matured (36), but are more likely to misfold during maturation (37) and the one mutant that has been tested is more subject to retrograde transport than wild-type PrP (29). Fourth, when proteasome activity is compromised, PrP accumulates in the cytoplasm, with the mutant accumulating faster than wild-type (29). Finally, when treated with the same concentration of proteasome inhibitors, neuroblastoma cells die much more rapidly than other cultured cell types tested (29).

Applicants asked if the hypersensitivity of neuroblastoma cells to proteasome inhibitors might be related to the accumulation of PrP in their cytoplasm. Positive findings prompted them to create transgenic mice that expressed PrP in the cytoplasm in the absence of proteasome inhibition. Indeed, the simple appearance of PrP in the cytoplasm is strongly and selectively neurotoxic, establishing the first clear mechanism for transforming wild-type PrP into a neurotoxic species. These observations suggest a new and potentially common framework for seemingly diverse PrP neuropathies. Through two very different approaches, Applicants have shown that the accumulation of even small amounts of PrP in the cytoplasm is sufficient to kill neuronal cells in a highly selective manner. In cultured cells, cytoplasmic accumulation was initiated by proteasome inhibition (34). In mice, cytoplasmic expression of PrP was directed by a transgene lacking an ER signal sequence. Here, three observations establish that pathology is directly attributable to transgene expression: 1) the same pathology was observed in two independent transgenic lines in a dosage-dependent manner, 2) Purkinje cells were spared, as expected from the known expression pattern of the promoter employed (56), and 3) pathology closely mimics that of transgenic mice with similar expression constructs producing mutant forms of PrP (55, 56).

The work presented herein establishes the first clear mechanism by which wild-type PrP can be converted into a highly neurotoxic species. Combining these results with previous studies on the retrograde transport of PrP (29), Applicants posit the following potentially unifying framework for spontaneous and familial PrP-based pathologies. Misfolded PrP molecules are retrograde transported to the cytoplasm for degradation by the proteasome (29, 30). Mutant PrP is more likely to misfold and be subject to retrograde transport (29, 37). The remarkable efficiency of proteasomal degradation normally prevents toxic species from accumulating. When the proteasome's ability to degrade PrP is compromised, as might naturally occur with stress and aging, the increase in cytoplasmic PrP would kill the neuron. Very small quantities of soluble PrP are toxic, perhaps acting directly or through PrP cleavage products (produced by caspases or other specific mechanisms) to signal cell death pathways. The toxicity of PrP is so extreme as to suggest it is an evolved mechanism to kill neurons with PrP folding proteins, thereby reducing the risk that PrP will accumulate at sufficient levels to produce the disseminating PrP^{Sc} form.

This model may not account for all PrP associated neuropathies, but it provides a simple explanation for several forms of the disease that would otherwise appear to have disparate etiologies. For example, it is compatible with the hypothesis that transmembrane forms of PrP are neurotoxic (25), but suggests their toxicity arises from the ability of the cell's quality-control system to recognize them as aberrant and shunt them to the cytoplasm (35). Indeed, mutations associated with neurodegeneration are distributed throughout the PrP coding sequence (9) and these proteins have been reported to accumulate in various compartments and perturb metabolism in a variety of ways (37). Each mutant might produce disease by an entirely different mechanism. However, it is much simpler to postulate a common mechanism: increased misfolding during maturation and recognition by the ER quality control system.

This work, together with an earlier study (36), establishes a clear mechanism by which wild-type PrP can be converted into a highly neurotoxic species. Misfolded PrP molecules are retrograde transported to the cytosol for degradation by the proteasome (36, 37). The remarkable efficiency of proteasomal degradation normally prevents toxic species from accumulating, but when the proteasome's ability to degrade PrP is compromised, as might naturally occur with stress and aging, the increase in cytosolic PrP would kill the neuron. Depending upon the rate of misfolding and retrograde transport, the same mechanism might lead to the production of PrP^{Sc} (53), but this is not the toxic species. Very small quantities of soluble PrP are toxic, perhaps acting directly or through PrP cleavage products (produced by caspases or other specific mechanisms) to signal cell death pathways. Indeed, the toxicity of cytosolic PrP is so extreme as to suggest it is an evolved mechanism to kill neurons with PrP folding problems, thereby reducing the risk that PrP will accumulate at sufficient levels to produce the disseminating PrP^{Sc} form.

Applicants' results suggest a unifying model for PrP-associated diseases that would otherwise appear to have disparate etiologies. Mutations associated with neurodegeneration are distributed throughout the PrP coding sequence (9) and these proteins have been reported to accumulate in different compartments and perturb metabolism in a variety of ways (52, 40-42). While accepting that these differences may modify disease progression, it seems simpler to postulate that a common mechanism underlies toxicity: the mutations increase misfolding of PrP, recognition by the cellular quality control systems, and transport to the cytosol. Such a mechanism might even explain pathogenesis in infectious prion diseases, if PrP^{Sc} induces perturbations in the folding and trafficking of endogenous PrP. In all of these cases, the low levels of soluble PrP required for toxicity would hitherto have eluded detection. This model would also resolve controversies about why some PrP mutations generate PrP^{Sc} and others do not (15, 27, 28, 43, 44). Cytosolic conversion of PrP depends on its rate of appearance in the cytosol (53), and may also be influenced in that compartment by the nature of the mutation (45).

Work described herein has implications for the use of proteasome inhibitors in biomedical research and as therapeutic agents (46, 47). Because small quantities of cytosolic PrP can cause severe neurodegeneration and because interfering with proteasome degradation leads to the accumulation of cytosolic PrP, proteasome inhibitors should be handled with caution, with strong preference given to inhibitors that do not cross the blood/brain barrier. Finally, alterations in PrP trafficking, such as that observed here with moPrP cells (49-50), can prevent toxic accumulation of PrP in the cytosol without compromising viability. This provides a potential therapeutic strategy for prion disease. In a related manner, Applicants' model explains the puzzling ability of certain neuroblastoma lines to continuously produce PrP^{Sc} without dying. In these cells, PrP^{Sc} conversion appears to occur solely on the cell surface and endocytic compartments.

In certain embodiments of the invention, the invention provides expression vectors comprising a nucleic acid sequence encoding a PrP polypeptide and a transcriptional regulatory sequence operably linked to the nucleotide sequence. A transcriptional regulatory sequence comprises at least one of a transcriptional promoter or transcriptional enhancer sequence, which regulatory sequence is operably linked to the PrP sequence. In certain embodiments of the invention, the transcriptional regulatory sequence operably linked to the nucleic acid sequence encoding a PrP polypeptide is an ecdysone-inducible promoter. In certain embodiments of the invention, the transcriptional regulatory sequence operably linked to the nucleic acid sequence encoding a PrP polypeptide is a tetracycline-inducible promoter. Tetracycline-inducible promoters include tet off promoters, with which, for example, expression of the PrP polypeptide is induced upon removal of tetracycline from the cell culture media and tet on promoters, with which, for example, expression of the PrP polypeptide is induced upon addition of tetracycline to the cell culture media. Any of a wide variety of expression control sequences that control the expression of a DNA sequence when operatively linked to it may be used in these vectors to express DNA sequences encoding a PrP polypeptide. In another embodiment, the nucleic acid may be included in an expression vector capable of replicating in and expressing the encoded PrP polypeptide in a prokaryotic or eukaryotic cell, such as a neuronal cell. In a related embodiment, the invention provides a host cell (e.g., mouse neuroblastoma N2A cells, PC12 cells) transfected with the expression vector.

A recombinant PrP nucleic acid can be produced by ligating the cloned gene, or a portion thereof, into a vector suitable for expression in either prokaryotic cells, eukaryotic cells, or both. Certain mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art.

In screening assays of the invention to identify drugs that inhibit the presence and/or accumulation of PrP, PrP^{Sc} or both as well as to identify drugs that inhibit the formation of pathological conformations of PrP and/or the toxicity of PrP in mammalian cells, the effect of a candidate drug may be assessed by, for example, assessing the effect of the candidate drug on kinetics, steady-state and/or endpoint of the reaction.

In additional embodiments of the invention, method formats include assays such as cell-based assays which utilize intact cells, such as neuroblastoma cells. Drugs to be tested can be produced, for example, by bacteria, yeast or other organisms (e.g., natural products), produced chemically (e.g., small molecules, including peptidomimetics), or produced recombinantly.

Assaying for drugs as described above, in the presence and absence of a candidate inhibitor, can be accomplished in any vessel suitable for containing the reactants. Examples include microtitre plates, test tubes, and micro-centrifuge tubes.

In an additional embodiment of the invention, a (one or more than one) drug that inhibits the presence and/or accumulation of PrP, PrP^{Sc} or both as well as a (one or more than one) drug that inhibits the formation of pathological conformations of PrP and/or the toxicity of PrP in mammalian cells is administered to an individual. The individual can be a mammal such as a human or a mouse. When administered to an individual, the drug can be administered as a pharmaceutical composition containing, for example, the drug and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. One skilled in the art would know that the choice of a pharmaceutically acceptable carrier, including a physiologically acceptable compound, depends, for example, on the route of administration of the composition.

One skilled in the art would know that a pharmaceutical composition containing a drug identified by or for use in an embodiment of the invention can be administered to a subject (e.g., a human or a transgenic mouse) by various routes including, for example, oral administration; intramuscular administration; intravenous administration; anal administration; vaginal administration; parenteral administration; nasal administration; intraperitoneal administration; subcutaneous administration and topical administration. The composition can be administered by injection or by intubation. The pharmaceutical composition also can be a drug linked to a liposome or other polymer matrix. Liposomes, for example, which consist of phospholipids or other lipids, are nontoxic, physiologically acceptable and metabolizable carriers that are relatively simple to make and administer.

A transgenic animal of the invention (e.g., a transgenic mouse) is any animal, preferably a non-human mammal, bird or an amphibian, in which one or more of the cells of the animal contain heterologous nucleic acid introduced by way of human intervention, such as by transgenic techniques well known in the art. In general, transgenic animal lines can be obtained by generating transgenic animals having incorporated into their genome at least one transgene, selecting at least one founder from these animals and breeding the founder or founders to establish at least one line of transgenic animals having the selected transgene incorporated into their genome.

The present invention is illustrated by the following examples, which are not intended to be limiting in any way.

The following materials and methods were used in Examples 1 and 2.

Materials and Methods:

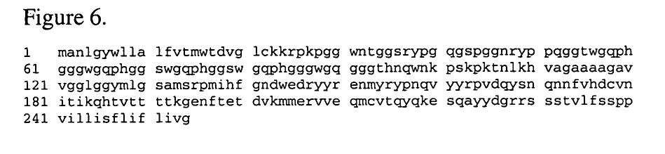

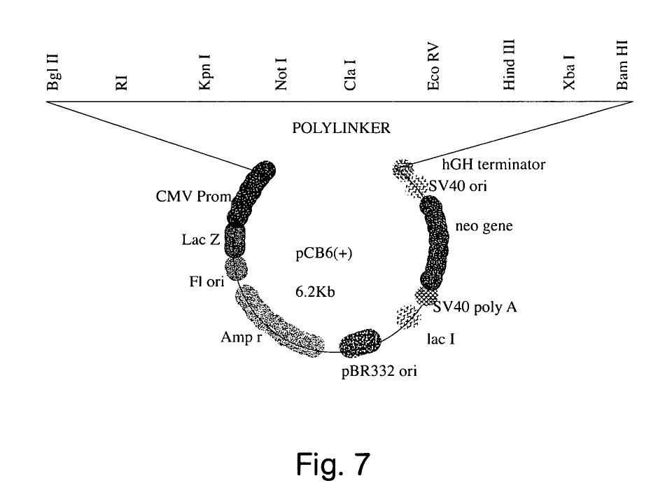

The PrP used in the examples in mouse PrP. The full length nucleic acid sequence (SEQ ID NO: 1, FIG. 5) and corresponding amino acid sequence (SEQ ID NO: 2, FIG. 6) of mouse PrP are found in GenBank, accession number NM 011170.

Cell culture and transfection. COS-1 cells were maintained in DMEM (Gibco BRL) with 10% fetal bovine serum. N2A mouse neuroblastoma cells (59) were cultured in OptiMEM (Invitrogen) with 10% fetal bovine serum. The PrP used in this study carried the hamster 3F4 epitope to facilitate detection. The pCB6+ vector was used in transfecting PrP. The pCB6+ vector is a pBR322-derived vector and includes a pBR322 origin of replication, a CMV promoter, a growth hormone termination sequence, and a Neomycin selection marker. Transfections were carried out using Fugene 6 (Roche) for COS cells and Lipofectamine (Invitrogen) for N2A cells. Pilot experiments were performed to ensure modest levels of PrP expression. To ensure equivalent levels of transfection when comparing cells transfected with wild-type and mutant PrP, a β-galactosidase expressing plasmid was co-transfected into both lines. To generate stable neuroblastoma cell lines, transfected N2A cells were bulk-selected by G418 (Invitrogen) and expression of PrP with the 3F4 epitope was verified by immunoblot and immunofluorescence analyses.

Proteasome inhibitor treatment. 24 hours after transfection, culture media was replaced with media containing proteasome inhibitors. 10 μM lactacystin (Calbiochem), 50 μM MG132 (Calbiochem), or epoxomicin (Affiniti) at indicated concentrations was added to the culture media. Cells were cultured at 37° C. with 5% CO2 for 16 hours, unless otherwise indicated. For transient proteasome inhibition, 50 μM MG132 or DMSO alone was added to the culture media 24 hrs after transfection, and cells were incubated at 37° C. for 2 hours. The media was removed and the cells washed with phosphate buffered saline (PBS) three times. Cells were then cultured in regular media for various times, as indicated. Cells co-transfected with PrP and CFTR plasmids were treated with 1 or 5 μM of MG132 for 2 hrs and harvested either immediately or after 12 hours of recovery in inhibitor-free medium.

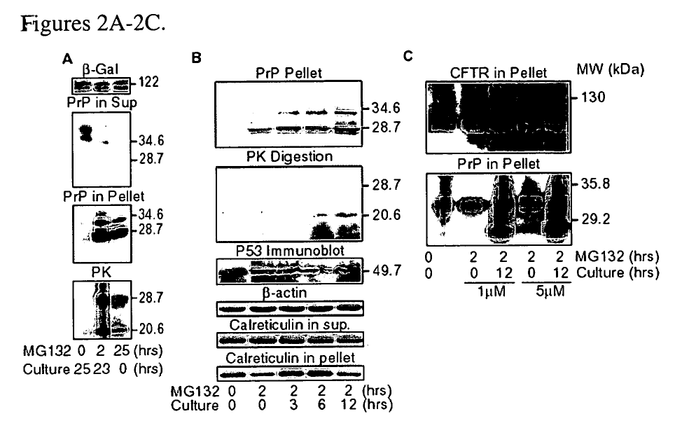

Analysis of PrP aggregation and proteinase K digestion. After transfection and treatment, cells growing in 6-well plates were washed once with ice-cold PBS and lysed with 300 μl (per well) of lysis buffer (50 mM Tris-HCl, pH 7.5, 150 mM NaCl, 2 mM EDTA, 0.5% (v/v) Triton X-100, 0.5% (w/v) Sodium Deoxycholate) on ice. Cells were disrupted by sequential passage through 21- and 25-gauge needles (10 times each) on ice. Fifty microliters of lysate was sedimented at 16,000 g for 30 minutes at 4° C. Supernatant proteins were precipitated with 4 volumes of 100% methanol (−20° C.) and incubated at 20° C. for at least 30 minutes. Both the pellet fraction and precipitated supernatants were sonicated in SDS-PAGE sample buffer containing 5% (w/v) SDS. For proteinase K (PK) digestions, 95 μl of lysate was incubated with PK at 37° C. for 30 minutes in a 100 μl reaction. The final PK concentration was 20 μg/ml (10 μg/ml for FIG. 2A). Digestions were terminated by adding 4 mM of Pefabloc SC (Roche) and incubating the samples on ice for 5 minutes. Proteins were precipitated by methanol and resolublized as described above. Proteins were analyzed on 14% or 16% SDS polyacrylamide gels, transferred to PVDF membrane and reacted with either 3F4 antibody at 1:5000, R20 antibody at 1:1000, γ-tubulin antibody (Sigma) at 1:5000, or calreticulin antibody (Stressgen) at 1:5000 dilution.

### EXAMPLE 1

Immunoblot analysis showed that a strong increase in PrP accumulation occurred in response to each inhibitor in each cell type (FIG. 1A). The normal form of PrP, PrPC, is soluble in mild detergents. The PrP that accumulated after proteasome inhibition was mostly detergent-insoluble (FIG. 1A) and migrated with the 27 kDa unglycosylated form of PrP that has had its NH₂-terminal and COOH-terminal signal sequences removed by ER processing enzymes (5). This is the state expected for PrP that misfolds in the ER, is retrograde transported to the cytoplasm, and accumulates there when proteasome degradation is blocked (5). Unglyocosylated proteins are more likely to misfold in the ER and be subject to retrograde transport; in addition, glycosylated species that are retrograde transported are subject to cytoplasmic deglycosidases. In all cell types, immunofluorescent analysis (5) confirmed that the protein had accumulated in the cytoplasm.

PrP^{Sc} is distinguished from other aggregate forms of PrP by an unusual pattern of protease resistance: approximately the first 90 amino acids remain very sensitive to proteinase K (PK) digestion, while the rest are extremely resistant (12). When detergent lysates of unfractionated cells were digested with PK, some yielded a major 21 kDa PK-resistant fragment (FIG. 1B), the same size as the PK-resistant fragment of unglycosylated PrP^{Sc} (13). The size of this fragment and the presence of both 3F4 (amino acids 108-111) and R20 (amino acids 218-232) epitopes (FIG. 1C), confirmed that it had the same distinctive cleavage pattern as PrP^{Sc} (12). Also, like PrP^{Sc}, its resistance to digestion was remarkable. The 21 kDa PrP band remained after virtually all Coomassie stainable material had been digested away.

Surprisingly, the fraction of PrP that converted to this species reproducibly varied greatly, with even modest differences in culture conditions (FIG. 1B). For example, aliquots of cells treated with 5 μM epoxomicin yielded much more of the 21 kDa PK-resistant fragment than identical aliquots treated with 1 μM epoxomicin, even though both had accumulated similar quantitites of aggregated PrP (compare the samples in FIG. 1B left, with those immediately above them). Similarly, aliquots treated with 50 μM MG132 and 10 μM Lactacystin accumulated comparable levels of aggregated PrP, but the former consistently yielded much more of the 21 kDa PK-resistant band (FIG. 1B and FIG. 1A, middle). 50 μM MG132 has a stronger proteasome inhibiting activity than 10 μM Lactacystin. These treatments did not kill COS cells. In contrast, many N2A culture cells were killed by these treatments. However, death did not increase with conversion to the PrP^{Sc}-like form. The 16 hr treatments with proteasome inhibitors employed here (FIG. 1A) did not kill COS cells or NIH 3T3 cells, but did kill some N2A cells. Notably, death did not correlate with conversion to the PrP^{Sc}-like form. That is, cells died at the same rates with 1 μM and 5 μM epoxomicin.

Various models of prion formation suggest that conversion to a specific ordered, rather than an amorphous aggregate, disordered aggregate, requires a critical number of PrP molecules in a susceptible conformation interactive to form a nucleus (18-19). Either because the nucleus is formed from a subset of specific conformers, all of which must come together at the same time. This would impose a strong concentration dependence on conversion.

Indeed, higher rates of conversion correlated with higher initial rates of accumulation. In the first few hours of proteasome inhibition, PrP accumulated more rapidly with 5 μM epoxomicin than with 1 μM epoxomicin and accumulated more rapidly in cells treated with MG132 than with Lactacystin (FIG. 1D). (At later times, accumulation might be equalized by modest changes in synthesis or degradation.) Also, transiently transfected (TT) N2A cultures produced much less PrP than stably transfected cultures, but they consistently yielded much more of the PrP^{Sc}-like PK-resistance band. In FIG. 1, total-protein blots of stably transfected cells were exposed for one-tenth the time of transiently transfected blots, but PK digestion blots of stably transfected samples were exposed 10 times longer. This large difference in conversion efficiency presumably relates to the highly concentrated expression in a small number of cells that is characteristic of transient transfections: all stably transfected cells expressed the protein but at a lower level. A mutant, PrP^{D177N} that causes heritable and transmissible forms of prion disease exhibits a higher rate of misfolding in the ER and displays a higher rate of retrograde transport (5). In the experiment of FIG. 1E, both cultures were transiently transfected, and equivalent levels of transfection were confirmed by equivalent levels of β-galactosidase expression from a co-transfected plasmid (5). In cells transfected with either wild-type PrP or PrP^{D177N}, the protein accumulated after proteasome inhibition. A higher level of accumulation occurred with PrP^{D177N} and this was associated with a disproportionately greater yield of the 21 kDa PK-resistant PrP^{Sc}-like species (FIG. 1E).

### EXAMPLE 2

The seminal characteristic of PrP^{Sc} is that, in some way, it promotes conversion of additional PrP to the same conformation (1, 20). To determine if the PrP^{Sc}-like conformer that arises denovo after proteasome inhibition has this property, Applicants asked if a transient loss of proteasome activity would be sustained after activity was restored. Transiently transfected COS cells were incubated with the reversible inhibitor MG132 for just 2 hrs, rinsed and cultured in media without the inhibitor for 21 hrs. Using a fluorogenic substrate Z-Leu-Leu-Leu-AMC, proteasome activity was high in controls, undetectable after 2 hrs of MG132 treatment, and restored to >70% of control levels 12 hrs later.

Transient MG132 treatments (FIG. 2A) led to much greater accumulation of PrP than continuous treatments (probably because longer treatments reduce protein synthesis). At the end of the incubation, the aggregated PrP exceeded the total quantity of PrP present initially, indicating that newly synthesized PrP continued to convert even after proteasome activity was restored. In a detailed time course, PrP had only begun to accumulate during the inhibitor treatment, and the 21 kDa PrP^{Sc}-like PK digestion product was not yet detectable (FIG. 2B). But a process had been initiated that caused new PrP protein to continue misfolding. Only a fraction of the continuously accumulating PrP converted to the PrP^{Sc}-like form, while a substantial portion was in a form readily digested by PK. Thus, conversion is accompanied by the production of other misfolded forms.

The brief proteasome treatment did not cause general protein aggregation. No changes in fractionation were observed with P-actin, calreticulin (FIG. 2B) or Coomassie-stained total proteins. Moreover, a well-characterized proteasome substrate, the endogenous p53 protein (21), accumulated during proteasome treatment but disappeared as activity was restored.

Finally, Applicants compared PrP with cystic fibrosis transmembrane conductance regulator (CFTR), another membrane protein that is subject to proteasome degradation and aggregates in response to proteasome inhibitors (8, 22). Immediately after proteasome inhibition, CFTR accumulated at moderate levels in an aggregated, detergent-insoluble state. No increase occurred during recovery (FIG. 2C). In contrast, detergent-insoluble PrP was barely detectable immediately after inhibition (FIG. 2C), but continued to accumulate after the inhibitor was removed.

The following materials and methods were used in Examples 3-6.

Materials and Methods:

The PrP used in the examples in mouse PrP. The full length nucleic acid sequence (SEQ ID NO: 1, FIG. 5) and corresponding amino acid sequence (SEQ ID NO: 2, FIG. 6) of mouse PrP are found in GenBank, accession number NM_{—}011170.

Cell culture and transient proteasome inhibition. N2A mouse neuroblastoma cells (59) were cultured in OptiMEM (Invitrogen) with 10% fetal bovine serum. To generate stable neuroblastoma cell lines, transfected N2A cells were bulk-selected with G418 (Invitrogen). The pCB6+ vector was used in transfecting PrP. The pCB6+ vector is a pBR322-derived vector and includes a pBR322 origin of replication, a CMV promoter, a growth hormone termination sequence, and a Neomycin selection marker. Expression of PrP containing the 3F4 epitope was verified by immunoblot and immunofluorescence analyses. Transient proteasome inhibitions were performed by including MG132 (50 μM), a reversible proteasome inhibitor, in culture media for 16 hrs or as indicated. After washing with PBS, cells were cultured in regular media for various indicated times. After treatment, cells growing on 6-well cell-culture plates were lysed with lysis buffer (50 mM Tris HCl, pH 7.5, 150 mM NaCl, 2 mM EDTA, 0.5% (v/v) Triton X-100, 0.5% (v/v) Sodium Deoxycholate) on ice. Cells were disrupted by sequential passages through 21- and 25-gauge needles 10 times on ice. Lysates were sedimented at 16,000 g for 30 min at 4° C. Proteins from supernatants were precipitated with methanol. Pellet fraction and precipitated supernatant were sonicated in SDS-PAGE sample buffer containing 5% (w/v) SDS and subject to electrophoresis. PrP protein was detected by immunoblot analysis with 3F4 antibody (Signet).

Ecdysone-inducible expression. Wild-type PrP (aa 1-254) or the cytoplasmic form of PrP (cyPrP, aa 23-230) were cloned into an ecdysone-inducible gene expression system (Invitrogen), which includes a pIND vector and pVgRXR plasmid. Plasmids carrying wild-type PrP or cyPrP and the Ecdysone receptor were transfected into N2A cells. Stably transfected cells were selected by G418 (Invitrogen) and Zeocin (Invitrogen). To induce PrP expression, 10 μM Pronasterone A (Invitrogen) was included in the culture media for 24 hrs. Analyses of cells were performed as described above.

TUNEL assay. Cells were grown on glass coverslips and treated with 50 μM MG132 for various indicated times. TUNEL staining was performed by using in situ cell death staining kit (Roche) in accordance with manufacturer's direction. Nuclei were stained with DAPI.

Pathological analysis of mouse tissue. Mouse tissues were dissected and immediately fixed in 4% paraformaldehyde and subjected to paraffin or epon embedding. Embedded tissues were sectioned, deparaffined, rehydrated and subjected to Haemotoxylin and Eosin staining or EM analysis.

RNase protection. Total RNA was isolated from various mouse tissues by using RNA STAT 60 (Tel-Test). RNase protection assay was performed with [32P]UTP-labeled anti-sense RNA (obtained through in vitro transcription of the anti-sense strand of PrP 3′ open reading frame corresponding to aa 193-254). Total RNA was used to hybridize with labeled anti-sense RNA followed by RNase One digestion (Invitrogen). Protected bands were electrophoresed and exposed to a phosphoimage screen. Scanning and quantification were performed by Storm 860 Scanner (Molecular Dynamics).

Footprint of mice. Footprints were recorded by dipping the mouse feet in India Black ink and allowing the mice to walk on a sheet of white paper.

### EXAMPLE 3

### Cytoplasmic Accumulation of PrP is Toxic to Neuroblastoma Cells

Applicants first asked if the toxicity of proteasome inhibitors in neuroblastoma cells is related to the accumulation of PrP in their cytosol. They compared closely related lines, derived from murine N2A cells. WtPN2A cells (also referred to as SecN2A cells) were produced by transfection with a plasmid expressing wild-type PrP (wtPrP, aa 1-254) from a constitutive promoter, CMV promoter. Bulk selection produced a pool of cells expressing PrP at different levels. By immunofluorescent staining and other analyses, we determined that the protein was localized at the surface in all cells (experiments were performed as in ref. 36). Cytosolic accumulation of PrP was induced by treatment with the reversible proteasome inhibitor MG132 and confirmed by immunofluorescent staining (experiments were performed as in ref. 36).

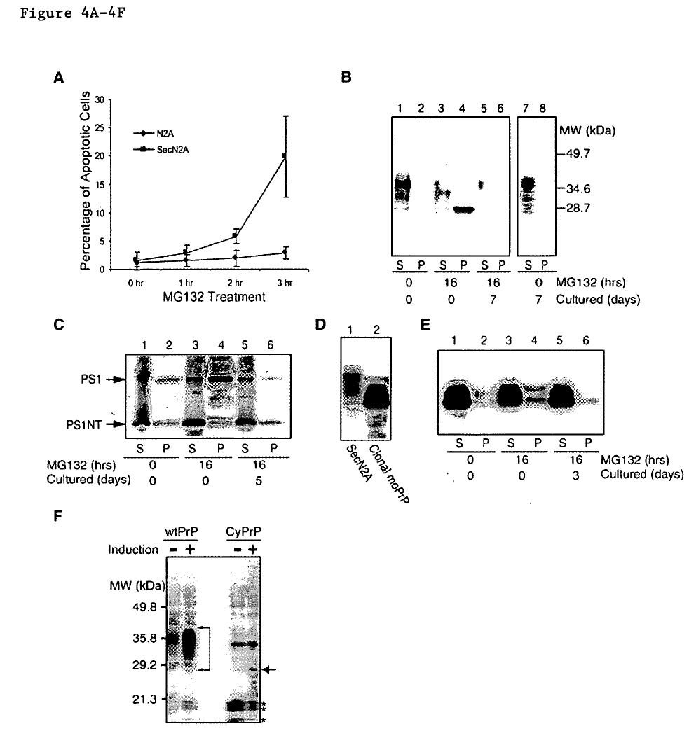

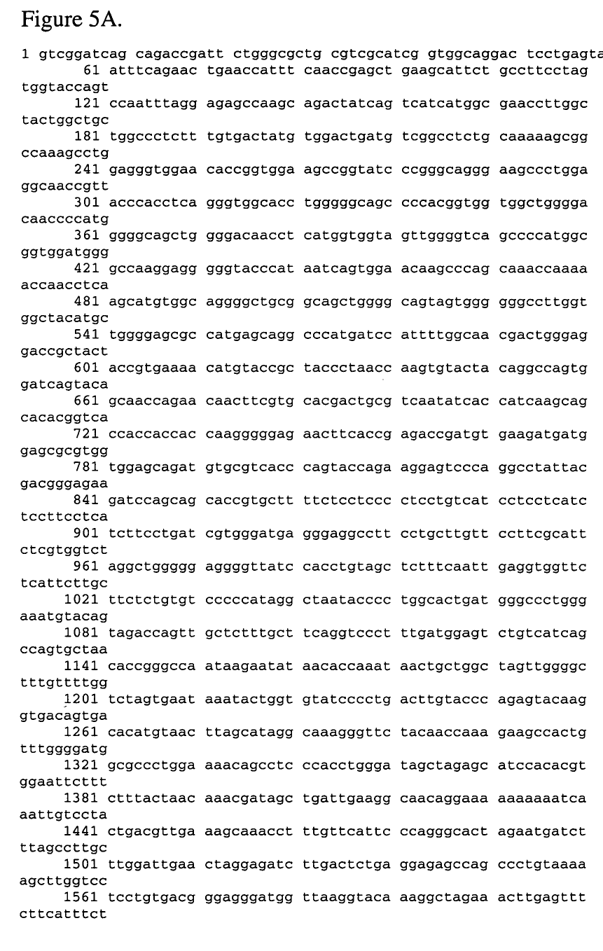

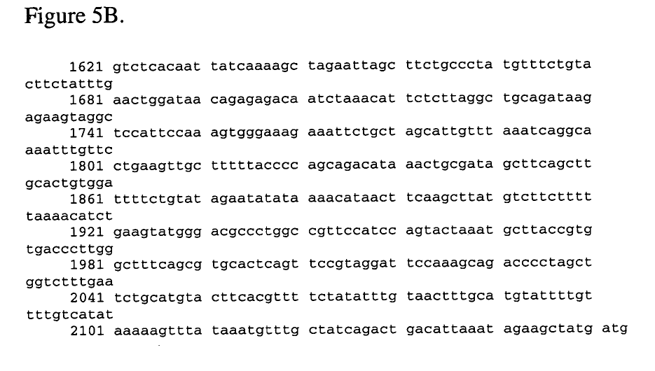

To determine if the hypersensitivity of neuroblastoma to proteasome inhibition is related to the amount of PrP accumulated in the cytosol, TUNEL assays were performed on murine neuroblastoma N2A cells and WtPN2A cells treated with or without MG132 for 3 hrs. Apoptotic cells were identified by the TUNEL assay and nuclei were stained with DAPI. Prior to proteasome inhibition, WtPN2A and N2A cultures were equally viable; during inhibition WtPN2A cells died much more rapidly (FIG. 4A). Seven days of regrowth in inhibitor-free medium were required to restore WtPN2A cultures to near confluence.

Before proteasome inhibition, PrP exhibited a normal heterogeneous pattern of glycosylation and fractionated in the supernatant after detergent lysis and centrifugation (FIG. 4B). Immediately after inhibition, much of the PrP fractionated in the pellet and migrated as expected for retrograde-transported PrP (36) (FIG. 4B), with its signal sequences removed, in a mostly unglycosylated form (54-55). Unglycosylated proteins are more likely to misfold in the ER and be subject to retrograde transport; glycosylated species that are retrograde transported are subject to cytoplasmic deglycosidases. After regrowth, PrP resumed its normal pattern of modification and localization. Although selection for the transgene was maintained continuously, cells that re-populated the culture produced much lower levels of PrP (FIG. 4B). A similar loss of PrP expression occurred with a variety of other proteasome inhibition-and-recovery protocols, including even very brief MG132 treatments (e.g. 3 hrs). Without the MG132 treatment, WtPN2A cells retained their original levels of PrP expression (FIG. 4B). Thus, cells with higher levels of PrP expression were selectively killed by treatment with the inhibitor.

In contrast, MG132 caused no selective killing in cells transfected with a plasmid encoding presenilin1, another membrane-associated protein that traffics through the ER and is subject to retrograde transport (56). When these cells were treated with the inhibitor, presenilin1 (like PrP) accumulated in the cytosol (56) in a detergent insoluble form (FIG. 4C). However, the cells died at about the same rate as the parental N2A line, resumed growth rapidly when MG132 was removed, and retained high levels of presenilin expression after regrowth (FIG. 4C). PS1 and PrP were detected by immunoblot analysis with an anti-PS 1 antibody or anti-PrP 3F4 antibody. Coomassie staining demonstrated equal loading of all supernatant and all pellet fractions except that lanes 3 and 4 of FIG. 4B were slightly underloaded. In all panels, PS1 and PrP were detected by immunoblot analysis with an anti-PS 1 antibody or the 3F4 antibody.

To address the importance of cytosolic localization of PrP in determining toxicity, Applicants examined a line that had been clonally selected for high constitutive PrP expression, moPrP (57). The PrP protein produced by these cells had an altered pattern of glycosylation (FIG. 4D), suggesting it was subject to a different pattern of intracellular trafficking (58). Indeed, when moPrP cells were treated with MG132, little PrP aggregated and by immunofluorescent staining none was accumulated in the cytosol (FIG. 4E). Although these cells expressed PrP at a very high level, they retained viability in the presence of MG132 even better than parental N2A cells, returned to confluence very quickly, and retained their high levels of PrP expression (FIG. 4E). More than 50% of N2A cells died within 12 hrs of proteasome treatment, but less than 5% of MoPrP cells died during the same period.

Finally, Applicants asked if increasing the appearance of PrP in the cytosol was sufficient to kill cells in the absence of proteasome inhibitors. Murine fibroblast-derived NIH3T3 cells were compared with neuroblastoma cells. Each cell line was separately transfected with wtPrP and with a cytosolic form (cyPrP, aa 23-230), which precisely eliminated the NH2-terminal and the COOH-terminal sequences that are cleaved upon ER entry. CyPrP has no cryptic ER translocation signals (31) and is unglycosylated, as is most retrograde transported PrP(36) (FIG. 4B). Using a constitutive promoter, CMV promoter, stable lines were readily established from NIH3T3 cells with both wtPrP and cyPrP. As expected, much less cyPrP accumulated than wtPrP because cyPrP was exposed to proteasomes directly after synthesis. Successful transfection and expression were confirmed by rapid accumulation of cyPrP upon addition of proteasome inhibitors. With neuroblastoma cells, stable lines were readily established with wtPrP, but could never be established with cyPrP, despite many attempts. Thus, cytosolic PrP appears to be toxic, but in a cell-type dependent manner.

For better transgene manipulation in neuroblastoma cells, Applicants expressed wtPrP and cyPrP from an ecdysone-inducible promoter. Again stable lines were readily established with wtPrP (FIG. 4F). Constitutive expression of wtPrP was higher than expected with this tightly controlled promoter (32), perhaps because the PrP coding sequence contains an enhancer for expression in this cell type (FIG. 4F). With this inducible promoter, lines could be established with cyPrP, but they grew very slowly. (This was likely due to leaky expression of the transgene, since stable lines were readily established with unrelated constructs). Immunofluorescence staining of cyPrP was faint but clearly above background. By immunoblotting, full-length cyPrP did not accumulate in quantities sufficient for detection, but specific cleavage fragments from the transgene did accumulate (FIG. 4F). Unlike cells transfected with wtPrP, cells transfected with cyPrP continuously yielded high levels of TUNEL-positive cells, indicating that even small amounts of cytosolic PrP, or perhaps its cleavage products, are toxic in N2A neuroblastoma cells. When expression was induced with ecdysone overnight, the number of TUNEL-positive cells doubled (from 6% to 14%), confirming the extreme toxicity of cyPrP. Apoptotic cells were stained with DAPI, 4′,6-diamidino-2-phenylindole.

### EXAMPLE 4

### Cytosolic PrP Produces Pathology Characteristic of Prion Disease in Transgenic Mice

To test if cytosolic PrP was toxic in a manner relevant to disease in whole animals, Applicants created transgenic mice expressing cyPrP from a commonly used PrP promoter (33, 34). The mPrP-1 vector, a minigene PrP vector for transgenic mice (33), was employed in generating transgenic mice herein described. Three founder mice carrying the transgene were identified by genomic PCR. One did not breed, developed hind limb paresis after 6 months, and died 4.5 months later. The other founders produced many transgenic progeny, all of whom exhibited pathology that was very different from that of transgenic mice neurologically impaired for other reasons, but very similar to that of transgenic mice producing mutant forms of PrP (34-35).

One founder, 2D1, exhibited no phenotype itself, but all of its transgenic offspring began to show an unsteady gait at 29 days (+/−2 days). Thereafter, they grew more slowly than wild-type siblings. Slow growth might be due to ataxia-associated problems with eating and drinking, although special care was taken to provide accessible food and water. At seven weeks, they were severely ataxic, very slow to respond to external stimuli and showed tail rigidity. 2D1 transgenic mice developed severe ataxia at 4 weeks after birth. Footprints of 6 weeks old wild-type and transgenic littermates revealed severe ataxia of transgenic mice. At 10 to 11 weeks, when death was obviously imminent as determined by the veterinarian, mice were euthanized.

Founder 1 D4 and its transgenic progeny developed disheveled hair and frequent scratching at 5 to 12 months of age. Mild ataxia and weight loss appeared several weeks later. F2 progeny carrying two copies of the transgene developed pathology with a much faster onset (about 2 months), demonstrating a dosage relationship between cyPrP and the pace of pathogenesis.

cyPrP transgenic mice exhibit ataxia and hair phenotypes. Photographs were taken of wild-type mouse, 1D4 transgenic mouse showing hair phenotype and 2D1 transgenic mice showing ataxia.

Detailed phenotypic changes of transgenic mice. Three founder mice carrying the PRP23-230 transgene were identified: 2D1, 1D4, and 3M. Founder 2D1 produced 41 transgenic progeny. Except for 19 pups sacrificed to analyze early pathological changes, all 22 transgenic progeny developed symptoms with an onset of 29+/−2 days. Founder 1D4 developed disheveled hair and frequent scratching at about 5 months of age. Approximately one month later, it lost hair on its neck and side due to scratching. Founder 1D4 produced 8F1 transgenic progeny before it was sacrificed at ˜6.5 months of age. Two of them were sacrificed at about 6 months of ages without any symptoms. Pathology analysis revealed cerebellar granular cell degeneration in both mice. The other 6 F1 transgenic progeny all developed disheveled hair and scratching, followed by mild ataxia and weight loss. Symptomatic onset was more variable in 1D4, occurring 5 to 12 months after birth. Mating between F1 transgenic 1D4 progeny produced some F2 transgenic progeny with a much more rapid and synchronized onset (about 2 months) of symptoms similar to that of 2D 1 transgenic mice. Southern blot analysis revealed these mice were homozygous for the transgenic. Founder 3M did not breed, developed hind limb paresis after 6 months, and died 4.5 months later.

Detailed pathological changes of transgenic mice. To investigate the anatomical and histological pathologies associated with disease, brains from F1 transgenic progeny of 2D1 and 1D4 founders were compared with age-matched wild-type littermates. By standard dissection and anatomical analysis, the only noticeable difference between transgenic and wild-type mice was atrophy of the cerebellum, which was obvious in transgenic 2D1 progeny sacrificed at 7 weeks. Atrophy was subtler in 1D4 mice.

Upon dissection, the only overt sign of disease in 2D1 and 1D4 mice was cerebellar atrophy. Histology revealed very similar cerebellar pathology with a timing and severity that corresponded to the onset and progression of ataxia. Massive neuronal loss occurred in the granular layer (G) and the molecular layer was also affected. Notably, Purkinje neurons (P) located between the molecular and granular layers, were unaffected. Purkinje cells are not spared in natural forms of prion diseases, however, the PrP promoter we used lacked an enhancer element required for expression in this cell type (35, 38). Thus, pathology was cell autonomous and related to transgene expression.

Severe gliosis in the cerebellum was revealed by immunohistochemical staining with an antibody against glial fibrilary acidic protein (GFAP) in both 2D1 and 1D4 mice. At early stages, behavior, brain morphologies and the timing of granular neuron migration were indistinguishable in transgenic mice and wild-type littermates. Therefore, pathology was due to degeneration rather than to problems in development.

### EXAMPLE 5

### Cerebellar Neuronal Degeneration in Transgenic Mice

By standard dissection, the only overt difference between the transgenic and wild-type mice was atrophy of the cerebellum. This was obvious in 2D1 progeny sacrificed at 7 weeks. Atrophy was more subtle in 1D4 mice.

Neuropathology of transgenic mice was demonstrated. Haemotoxylin and Eosin (HE) staining of cerebella of 7-week-old wild-type and transgenic 2D 1 littermates was conducted. GFAP immunostaining of adjacent sections with haemotoxylin counter staining was also conducted. HE staining of cerebella of wild-type and transgenic 2D 1 littermates from 9-days-old to 5-weeks-old, and HE staining of cerebella of wild-type and transgenic 1D4 littermates was conducted as well.

Paraffin- and epon-embedded tissues revealed similar changes in the cerebellar cortex for both 2D1 and 1D4 mice with a timing that corresponded to the onset of their symptoms. In 7-week-old transgenic 2D1 mice, almost all neurons in the granular layer had disappeared, leaving a large number of intracellular vacuoles and extracellular space within the neuropil. The molecular layer (M) appeared moderately narrowed. Intracellular vacuoles and extracellular spaces within the neuropil were also observed within the molecular layer. Neurons between the molecular and granular layers, Purkinje cells (P) were apparently not affected. Severe gliosis in the cerebellum region was revealed by immunohistochemical staining with an antibody against glial fibrilary acidic protein (GFAP).

To determine whether neuropathology was due to problems in development or to neurodegeneration, we sacrificed pairs of transgenic 2D1 mice and their wild-type littermates at different ages. Early in postnatal development, brain morphologies and the timing of granular neuron migration were indistinguishable in wild-type and transgenic mice. No difference in the amount and timing of granular cell migration occurred between wild-type and transgenic littermates at 9 days and 3 weeks after birth. However, in transgenic mice granular neurons subsequently degenerated. In 2D1 mice, degeneration proceeded very rapidly. Granular cells in transgenic mice degenerated rapidly after 3 weeks. By 9 weeks, the entire granular layer of neurons had disappeared. Normal development, followed by neurodegeneration, roughly coinciding with the onset of behavioral symptoms, was also observed in 1D4 mice.

The very predictable onset of pathology in 2D1 mice made it possible to perform ultrastructural analysis prior to overt neurodegeneration. Electron micrographs were prepared of granular neurons in the cerebella of 3-week-old wild-type and transgenic 2D1 littermates. High magnification revealed these vacuoles were derived from swollen mitochondria. Large vacuoles appeared in the granular neurons of transgenic mice at three weeks of age. At higher magnification, these appeared to be derived from swollen mitochondria, with fragmented cristae often visible. Some nuclei exhibited striking condensation and fragmentation, suggestive of apoptosis. The same morphology was observed in all 2D1 (6 samples) and 1D4 (1 sample, 6 months of age) transgenic mice analyzed, but in none of their non-transgenic littermates (7 samples), demonstrating that these changes were an early reflection of pathology, not an artifact of fixation.

No compromise in protein folding or quality control in transgenic mice. To ensure the phenotypic changes in transgenic mice were not due to cyPrP-overwhelmed protein folding or quality control mechanisms, Applicants compared wild-type and transgenic brain tissues. No indication of general problems in these systems was found. Wild-type and transgenic brain tissues showed 1) no differences in proteasome activity assayed with a fluorogenic peptide substrate, 2) no induction of Hsp7O, BiP, or other general markers of protein folding stress detectable by immunoblotting, and 3) no increase in aggregation of any cellular proteins detectable by Coomassie staining after differential centrifugation.

### EXAMPLE 6

### Low Levels of Cytosolic PrP are Sufficient to Kill Neurons

To determine the copy number of integrated transgene, Southern Blot analysis on 1D4 mice was performed. Mouse tail genomic DNA was digested with EcoRI and probed with PrP coding sequences. Seven copies of the transgene were integrated into 1D4 mice. The F2 progeny that showed rapid and synchronized onset of symptoms was homozygous for the transgene.

PrP is widely expressed, but prion disease pathologies diseases are generally restricted to the central nervous system (9). The toxicity of the transgene recapitulated this tissue-selectivity. Expression of transgene was demonstrated. RNA expression for endogenous PrP and transgene (PrP23-230) in different tissues of two pairs of wild-type and transgenic littermates was shown. Heart, liver, spleen, brain, and skeletal muscle were looked at. Immunoblot analysis of PrP expression in brain, and in heart was conducted. To determine the expression levels of transgene in different tissues, an RNase protection assay was performed in wild-type and transgenic littermates. Quantification revealed that the transgene was expressed in the brain at about 1.2 fold the level of endogenous PrP. In heart and skeletal muscle, the transgene was expressed at relatively high levels when compared to endogenous PrP. As expected (9), using an RNase protection assay (32), Applicants showed that endogenous PrP was expressed at high levels in the brain and at modest levels in heart and skeletal muscle. In 2D 1 mice, transgene expression was similar to endogenous PrP in the brain and at slightly higher levels in heart and muscle. Thus, the extreme pathology in the brains of transgenic mice and the absence of any detectable pathology in heart and muscle recapitulated the tissue selectivity of prion disease.

Cross validation of selective toxicity was provided by separate studies with transfected cell lines. When 3T3 and neuroblastoma cells were transfected with a plasmid directing the expression of the mature form of PrP in the cytoplasm, stable lines were readily and repeatedly established for the 3T3 lines but never for the neuroblastoma line. Stable transformants of neuroblastoma cells could be obtained with other plasmids including those for full-length PrP (48).

Three PrP species were detected by immunoblot analysis in the brains of wild-type mice. These correspond to the characteristically abundant mono-glycosylated and di-glycosylated forms of PrP and the less abundant unglycosylated form (39). Because N-linked glycosylation takes place in the ER and our transgene lacked the N- and C-terminal signal sequences that are normally removed by ER processing enzymes, its protein product should migrate at the same position as the smallest form of endogenous PrP, 27 kDa.

Endogenous PrP signals were very strong in the brain. Using serially diluted proteins to ensure that film responses were in the linear range, ˜a 2-fold increase in the smallest (27 kDa) PrP band was detected in three out of three of the 2D 1 brain samples examined (48). That is, the total level of PrP accumulating from the transgene in the cytoplasmic compartment was roughly equivalent to the low level of endogenous unglycosylated PrP in the ER/secretory compartment, a small fraction of total wild-type PrP. Endogenous PrP expression was lower in the heart and wild-type PrP bands could barely be detected above background. Protein encoded by the 2D1 transgene was clearly, and reproducibly detectable in the heart by increased reactivity of a 27 kD species (48). In 1D4 mice, transgene RNA could be detected by RNase protection assay(48), but levels were lower than for 2D1 mice and the protein could not be reliably detected above background. These results confirm the selective toxicity of cytoplasmic PrP and establish that very small levels of its accumulation or its proteasomal breakdown products in this cellular compartment are sufficient to kill neurons in a dosage dependent manner.

The conformation of PrP associated with infectious PrP diseases, PrP^{Sc}, is detergent insoluble and yields a characteristic resistant fragment after cleavage by proteinase K (9). No PrP^{Sc}, nor any other forms of aggregated PrP, could be detected in 2D1 or 1D4 mice at any of several stages tested. PrP^{Sc} was readily detected in brain samples from infected hamster. The absence of pathology in Purkinje cells, also attested to the absence of PrP^{Sc}. Neurons immediately adjacent to Purkinje cells were subject to massive degeneration and an infectious agent would have been expected to spread to them.

Incorporation by Reference

All publications mentioned herein are hereby incorporated by reference in their entirety as if each individual publication was specifically and individually indicated to be incorporated by reference. In case of conflict, the present application, including any definitions herein, will control.

Equivalents

As those skilled in the art will appreciate, numerous changes and modifications may be made to the embodiments of the invention without departing from the spirit of the invention. It is intended that all such variations fall within the scope of the invention.

### REFERENCES

1. S. B. Prusiner, Proc Natl Acad Sci USA 95, 13363-83 (1998); C. Weissmann, Trends in Cell Biol. 4, 10 (1994); B. Caughey, B. Chesebro, Adv Virus Res 56, 277-311 (2001); J. Collinge, Annu Rev Neurosci 24, 519-50 (2001); A. Aguzzi, F. L. Heppner, Cell Death Differ 7, 889-902. (2000).

2. R. H. Kimberlin, Nature 297, 107-8. (1982);

3. A. G. Dickinson, G. W. Outram, Ciba Found Symp 135, 63-83 (1988).

4. J. Ma, S. Lindquist, Nat Cell Biol 1, 358-361 (1999).

5. J. Ma, S. Lindquist, Proc Natl Acad Sci USA 98, 14955-60. (2001).

6. Y. Yedidia et al, Embo J 20, 5383-91 (2001).

7. S. W. Fewell, K. J. Travers, J. S. Weissman, J. L. Brodsky, Annu Rev Genet 35, 149-91 (2001).

8. R. R. Kopito, Physiol Rev 79, S167-73 (1999);

9. S. B. Prusiner, Proc Natl Acad Sci USA 95, 13363-83 (1998).

10. B. Caughey, B. Chesebro, Trends in Cell Biol. 7, 56-62 (1997).

11. R. Chiesa, D. A. Harris, Neurobiol Dis 8, 743-63 (2001).

12. P. Parchi et al., Proc Natl Acad Sci USA 97, 10168-72 (2000).

13. J. Collinge, K. C. Sidle, J. Meads, J. Ironside, A. F. Hill, Nature 383, 685-90 (1996).

14. B. Caughey, D. A. Kocisko, G. J. Raymond, P. T. Lansbury, Jr., Chem Biol 2, 807-17 (1995).

15. A. Aguzzi, F. L. Heppner, Cell Death Differ 7, 889-902 (2000).

16. J. Collinge, Annu Rev Neurosci 24, 519-50 (2001).

17. H. Bueler et al., Cell 73, 1339-47 (1993).

18. P. T. Lansbury, Jr., B. Caughey, Chem Biol 2, 1-5 (1995).

19. T. R. Serio et al., Science 289, 1317-21. (2000).

20. D. A. Kocisko et al, Nature 370, 471-4 (1994).

21. L. Meng et al, Proc. Natl. Acad. Sci USA 96, 10403-8 (1999).

22. W. C. Wigley et al., J Cell Biol 145, 481-90 (1999).

23. S. Brandner et al., Nature 379, 339-43 (1996).

24. L. Ivanova, S. Barnada, T. Kummer, D. A. Harris, J Biol Chem 276, 42409-21 (2001).

25. K. K. Hsiao et al., Proc Natl Acad Sci USA 91, 9126-30 (1994).

26. C. I. Lasmezas et al., Science 275, 402-5 (1997).

27. T. Kitamoto, R. Iizuka, J. Tateisbi, Biochem Biophys Res Commun 192, 525-31 (1993).

28. F. Tagliavini et al., Cell 79, 695-703 (1994).

29. R. R. Kopito, Cell 88, 427-30 (1997).

30. S. W. Fewell, K. J. Travers, J. S. Weissman, J. L. Brodsky, Annu Rev Genet 35, 149-91 (2001).

31. C. Holscher, U. C. Bach, B. Dobberstein, J Biol Chem 276, 13388-94 (2001).

32. D. No et al., Proc Natl Acad Sci USA, 93, 3346-51 (1996).

33. D. R. Borchelt et al., Genet Anal 13, 159-63 (1996).

34. R. Chiesa, P. Piccardo, B. Ghetti, D. A. Harris, Neuron 21, 1339-51 (1998).

35. D. Shmerling et al., Cell 93, 203-14 (1998).

36. J. Ma, S. Lindquist, Proc Natl Acad Sci USA 98, 14955-60 (2001).

37. Y. Yedidia, L. Horonchik, S. Tzaban, A. Yanai, A. Taraboulos, Embo J 20, 5383-91 (2001).

38. R. Chiesa et al., Proc Natl Acad Sci USA 97, 5574-9 (2000).

39. M. Russelakis-Carneiro, G. P. Saborio, L. Anderes, C. Soto, J Biol Chem 18, 18 (2002).

40. H. Lorenz, O. Windl, H. A. Kretzschmar, J Biol Chem 277, 8508-16 (2002).

41. N. Singh et al., J Biol Chem 272, 28461-70 (1997).

42. G. Zanusso et al., J Biol Chem 274, 23396-404 (1999).

43. D. Doyle, M. Rogers, Trends Genet 14, 171-3 (1998).

44. P. Piccardo et al., J Neuropathol Exp Neurol 57, 979-88 (1998).

45. J. J. Liu, S. Lindquist, Nature 400, 573-6 (1999).

46. Adams, V. J. Palombella, P. J. Elliott, Invest New Drugs 18, 109-21 (2000); P. Andre et al., Proc Natl Acad Sci USA 95, 13120-4 (1998).

47. P. Andre et al., Proc Natl Acad Sci USA 95, 13120-4 (1998).

48. W. Swietnicki, R. B. Petersen, P. Gambetti, W. K. Surewicz, J Biol Chem 273, 31048-52 (1998).

49. B. Caughey, G. J. Raymond, Journal of Biological Chemistry 266, 18217-23 (1991).

50. K. Doh-Ura, T. Iwaki, B. Caughey, J Virol 74, 4894-7 (2000); B. Caughey, G. J. Raymond, Journal of Biological Chemistry 266, 18217-23 (1991).

51. S. Liemann, R. Glockshuber, Biochemistry 38, 3258-67 (1999).

52. L. Ivanova, S. Barmada, T. Kummer, D. A. Harris, J Biol Chem 276, 42409-21 (2001).

53. J. Ma, S. Lindquist, Science (2002).

54. A. J. Parodi, Annu Rev Biochem, 69, 69-93 (2000).

55. T. Suzuki, H. Park, N. M. Hollingsworth, R. Stemglanz, W. J. Lennarz, J Cell Biol 149, 1039-52 (2000).

56. J. A. Johnston, C. L. Ward, R. R. Kopito, J Cell Biol 143, 1883-98 (1998).

57. B. Chesebro et al., Developments in Biological Standardization 80, 131-40 (1993).

58. A. Helenius, M. Aebi, Science 291, 2364-9 (2001). 59. Olmsted J B, et al. Isolation of microtubule protein from cultured mouse neuroblastoma cells. Proc. Natl. Acad. Sci. USA 65: 129-136, 1970.

60. P. Picardo et al, J. Neuropathol Exp Neurol 57, 979-88, (1998).

61. G. S. Jackson et al, Science 283, 1935-7 (1999).

62. I. Mehlhom et al., Biochemistry 35, 5528-37 (1996).

63. S. Lehmann, D. A. Harris, J Biol Chem 272, 21479-87 (1997).

What is claimed is:

1. A method of identifying a drug that inhibits the formation of PrP in the cytoplasm of mammalian cells, comprising:
(a) culturing test cells that ectopically express PrP in the cytoplasm in the presence of a candidate drug;
(b) culturing control cells in the absence of the candidate drug of (a); and
(c) comparing the viability of the test cells of (a) with the control cells of (b), wherein, if the viability of the test cells of (a) is greater than the viability of the control cells of (b), the candidate drug is a drug that inhibits the formation of PrP in the cytoplasm of mammalian cells.

2. The method of claim 1, wherein the PrP in step (a) and step (b) is under the control of an inducible promoter.

3. The method of claim 1, wherein the mammalian cells are neuronal cells.

4. A method of identifying a drug that inhibits the accumulation of PrP^{Sc} in mammalian cells, comprising:
(a) culturing test cells that ectopically express PrP^{Sc} in the presence of a candidate drug;
(b) culturing control cells in the absence of the candidate drug of (a); and
(c) comparing the viability of the test cells of (a) with the control cells of (b), wherein, if the viability of the test cells of (a) is greater than the viability of the control cells of (b), the candidate drug is a drug that inhibits the accumulation of PrP^{Sc} in mammalian cells.

5. The method of claim 4, wherein the PrP^{Sc} in step (a) and step (b) results from ectopically expressed PrP under the control of an inducible promoter.

6. The method of claim 4, wherein the mammalian cells are neuronal cells.

7. A method of identifying a drug that inhibits the formation of PrP^{Sc} and PrP in the cytoplasm of mammalian cells, comprising:
(a) culturing test cells that ectopically express PrP^{Sc} and PrP in the cytoplasm in the presence of a candidate drug;
(b) culturing control cells in the absence of the candidate drug of (a); and
(c) comparing the viability of the test cells of (a) with the control cells of (b), wherein, if the viability of the test cells of (a) is greater than the viability of the control cells of (b), the candidate drug is a drug that inhibits the formation of PrP^{Sc} and PrP in the cytoplasm of mammalian cells.

8. The method of claim 7, wherein the PrP in step (a) and step (b) is under the control of an inducible promoter.

9. The method of claim 7, wherein the mammalian cells are neuronal cells.

10. A method of identifying a drug that inhibits the formation of a pathological conformation of PrP in mammalian cells, comprising:
(a) culturing test cells that ectopically express PrP in the cytoplasm in the presence of a candidate drug;
(b) culturing control cells in the absence of the candidate drug of (a); and
(c) comparing the viability of the test cells of (a) with the control cells of (b), wherein, if the viability of the test cells of (a) is greater than the viability of the control cells of (b), the candidate drug is a drug that inhibits the formation of a pathological conformation of PrP in mammalian cells.

11. The method of claim 10, wherein the PrP in step (a) and step (b) is under the control of an inducible promoter.

12. The method of claim 10, wherein the mammalian cells are neuronal cells.

13. A method of identifying a drug that inhibits improper processing of PrP in mammalian cells, comprising:
(a) culturing test cells that ectopically express PrP in the cytoplasm in the presence of a candidate drug;
(b) culturing control cells in the absence of the candidate drug of (a); and
(c) comparing the viability of the test cells of (a) with the control cells of (b), wherein, if the viability of the test cells of (a) is greater than the viability of the control cells of (b), the candidate drug is a drug that inhibits improper processing of PrP in mammalian cells.

14. The method of claim 13, wherein the PrP in step (a) and step (b) is under the control of an inducible promoter.

15. The method of claim 13, wherein the mammalian cells are neuronal cells.

16. A method of identifying a drug that inhibits PrP toxicity in mammalian cells, comprising:
(a) culturing test cells that ectopically express PrP in the cytoplasm in the presence of a candidate drug;
(b) culturing control cells in the absence of the candidate drug of (a); and
(c) comparing the viability of the test cells of (a) with the control cells of (b), wherein, if the viability of the test cells of (a) is greater than the viability of the control cells of (b), the candidate drug is a drug that inhibits PrP toxicity in mammalian cells.

17. The method of claim 16, wherein the PrP in step (a) and step (b) is under the control of an inducible promoter.

18. The method of claim 16, wherein the mammalian cells are neuronal cells.

19. A method of treating a prion disease in an individual, comprising administering a drug identified by the method of claim 1 to an individual, wherein the prion disease is treated in the individual.

20. A method of treating a prion disease in an individual, comprising administering a drug identified by the method of claim 4 to an individual, wherein the prion disease is treated in the individual.

21. A method of treating a prion disease in an individual, comprising administering a drug identified by the method of claim 7 to an individual, wherein the prion disease is treated in the individual.

22. A method of treating a prion disease in an individual, comprising administering a drug identified by the method of claim 10 to an individual, wherein the prion disease is treated in the individual.

23. A method of treating a prion disease in an individual, comprising administering a drug identified by the method of claim 13 to an individual, wherein the prion disease is treated in the individual.

24. A method of treating a prion disease in an individual, comprising administering a drug identified by the method of claim 16 to an individual, wherein the prion disease is treated in the individual.

25. The method of claim 24, wherein the drug is administered in combination with one or more of a drug which inhibits ectopic expression PrP in the cytoplasm of mammalian cells.

26. A transgenic nonhuman mammal ectopically expressing PrP in the cytoplasm of its cells.

27. The transgenic mammal of claim 26 which is a transgenic mouse.

28. A transgenic mouse expressing PrP, in the cytoplasm of its cells, from nucleic acids introduced into at least one cell from which the transgenic mouse or an ancestor thereof was produced.

29. The transgenic mouse of claim 28, wherein PrP is expressed constitutively.

30. The transgenic mouse of claim 29, wherein PrP is expressed under the control of an inducible promoter.

31. A method of identifying a drug that inhibits the effects of PrP present in the cytoplasm of cells, comprising:
(a) administering a candidate drug to a test animal, wherein the test animal is a transgenic nonhuman mammal that ectopically expresses PrP in the cytoplasm of its cells;
(b) assessing the effects of PrP on the test animal; and
(c) assessing the effects of PrP on a corresponding control animal, wherein if the effects of PrP in the test animal are less than the effects of PrP in the corresponding control animal, the candidate drug is a drug that inhibits the effects of PrP present in the cytoplasm of cells.

32. The method of claim 31, wherein the transgenic nonhuman mammal is a transgenic mouse.


SEQUENCE LISTING (2 sequences)

SEQ ID NO: 1 - DNA, 2153 bp, Mus musculus
gtcggatcagcagaccgattctgggcgctgcgtcgcatcggtggcaggactcctgagtatatttcagaactgaaccatttcaaccgagctgaagcattctgccttcctagtggtaccagtccaatttaggagagccaagcagactatcagtcatcatggcgaaccttggctactggctgctggccctctttgtgactatgtggactgatgtcggcctctgcaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccataatcagtggaacaagcccagcaaaccaaaaaccaacctcaagcatgtggcaggggctgcggcagctggggcagtagtggggggccttggtggctacatgctggggagcgccatgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagcagcaccgtgcttttctcctcccctcctgtcatcctcctcatctccttcctcatcttcctgatcgtgggatgagggaggccttcctgcttgttccttcgcattctcgtggtctaggctgggggaggggttatccacctgtagctctttcaattgaggtggttctcattcttgcttctctgtgtcccccataggctaatacccctggcactgatgggccctgggaaatgtacagtagaccagttgctctttgcttcaggtccctttgatggagtctgtcatcagccagtgctaacaccgggccaataagaatataacaccaaataactgctggctagttggggctttgttttggtctagtgaataaatactggtgtatcccctgacttgtacccagagtacaaggtgacagtgacacatgtaacttagcataggcaaagggttctacaaccaaagaagccactgtttggggatggcgccctggaaaacagcctcccacctgggatagctagagcatccacacgtggaattctttctttactaacaaacgatagctgattgaaggcaacaggaaaaaaaaaatcaaattgtcctactgacgttgaaagcaaacctttgttcattcccagggcactagaatgatctttagccttgcttggattgaactaggagatcttgactctgaggagagccagccctgtaaaaagcttggtcctcctgtgacgggagggatggttaaggtacaaaggctagaaacttgagtttcttcatttctgtctcacaattatcaaaagctagaattagcttctgccctatgtttctgtacttctatttgaactggataacagagagacaatctaaacattctcttaggctgcagataagagaagtaggctccattccaaagtgggaaagaaattctgctagcattgtttaaatcaggcaaaatttgttcctgaagttgctttttaccccagcagacataaactgcgatagcttcagcttgcactgtggattttctgtatagaatatataaaacataacttcaagcttatgtcttctttttaaaacatctgaagtatgggacgccctggccgttccatccagtactaaatgcttaccgtgtgacccttgggctttcagcgtgcactcagttccgtaggattccaaagcagacccctagctggtctttgaatctgcatgtacttcacgttttctatatttgtaactttgcatgtattttgttttgtcatataaaaagtttataaatgtttgctatcagactgacattaaatagaagctatgatg

SEQ ID NO: 2 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG